(19) United States
(12) Patent Application Publication
(10) Pub. No.: US 2006/0020037 A1
(43) Pub. Date: Jan. 26, 2006

(54) Tazarotenic acid and esters thereof for treating autism

(75) Inventor: Martin A. Voet, San Juan Capistrano, CA (US)

Correspondence Address: Robert J. Baran (T2-7H);ALLERGAN, INC., Legal Department, 2525 Dupont Drive, Irvine, CA, 92612, US

(73) Assignee: Allergan, Inc.

(21) Appl. No.: 10/898,534

(22) Filed: Jul. 22, 2004

(51) Int. Cl. A61K 31/192

(52) U.S. Cl. 514/569

(57) ABSTRACT

This invention provides a method of treating autism comprising administering a therapeutically effective amount of a compound selected from the group consisting of tazarotenic acid, or ester or salt, thereof, to a person in need of such treatment. Preferably said compound is tazarotene.

### BACKGROUND OF THE INVENTION

1. Field of the Invention

The present invention relates to the treatment of autism.

2. Description of the Related Art

Autism is a disabling neurological disorder that affects thousands of Americans and includes a number of subtypes, with various assumed causes and few documented ameliorative treatments. Autism is characterized by behavioral syndrome often recognized between two and three years of age. There is no clear-cut biological marker for autism. Diagnosis of the disorder is made by considering the degree to which the child matches the behavioral syndrome, which is characterized by poor communicative abilities, peculiarities in social and cognitive capacities, and maladaptive behavioral patterns.

There currently is no known medical treatment for autism. A number of different therapies have been attempted in an effort to cure autism or at least lessen its symptoms, including drug therapies as well as psychiatric care and attempted counseling. In general, results of such treatments have been disappointing, and autism remains very difficult to effectively treat, particularly in severe cases.

U.S. Pat. No. 6,585,996 B1 discloses the use of lipid-soluble thiamine derivatives in the treatment of autism.

U.S. Pat. No. 6,552,000 B2 discloses the use of certain anticonvulsant derivatives for treating autism.

U.S. Pat. No. 6,447,772 B1 discloses the use of one or both of a purified casomorphin inhibitor and a purified gluteomorphin inhibitor in the treatment of autism.

U.S. Pat. No. 5,008,251 discloses the use of purine nucleotides and derivatives, intermediates and analogs thereof for treating autism.

Megson proposes that the use of unsaturated “cis” vitamin A can improve the symptoms of autistic children by repairing damage to their retina.

Once you understand the way autistic children see their world, says Dr. Mary Megson, a professor of pediatrics at the Medical College of Virginia, “the fact that they don't look you in the eye and can't bear for things to be changed makes perfect sense.” She emphatically rejects the widely accepted hypothesis that these children have no theory of mind (i.e., no understanding that other people have their own thoughts, plans, points of view), and that they relate to other people as just another type of thing.

Instead, she maintains that their seemingly alienated behavior is perfectly rational. It is their way of surviving in an extraordinary and terrifying visual world, the result of damage to a protein pathway that affects the way that certain specialized cells in their retinas work. “Imagine that everything appeared to you like a some paintings by Picasso, flat and two-dimensional, with various features superimposed,” urges Dr. Megson, who has specialized in development disorders for the last 15 years. “Or think of a Hockney collage, digitally remastered with all the depth cues taken out.” (For reference to Dr. Megson's theories, see www.megson.com. In particular, see “The Biological Basis for Perceptual Defecits in Autism” at http://www.megson.com/Biological Basis/Biological Basis.html.)

Tazarotene is a RAR_{β} and RAR_{δ}-selective retinoid agonist which has been used for treating psoriasis and/or acne. (See U.S. Pat. No. 5,089,509.) Tazarotene and other related retinoids are disclosed for treating various other diseases and conditions which are responsive to treatment with retinoid compounds. (See U.S. Pat. Nos. 5,750,693; 6,090,826 and 6,344,463.) Also, it has recently been disclosed that tazarotene and certain other retinoid agonists are useful in preventing the proliferation of retinal pigment epithelium following surgery or trauma or resulting from ocular diseases associated with choroidal neovascularization, such as age-related macular degeneration and histoplasmosis syndrome. (See U.S. Pat. Nos. 5,824,685; 6,075,032; 6,071,924; 6,372,753; 5,437,291 and 5,674,205.)

### SUMMARY OF THE INVENTION

The present invention provides a method of treating autism comprising administering a therapeutically-effective amount of a compound selected from the group consisting of tazarotenic acid, salts and esters thereof and mixtures of said acid, salts and esters to a person in need of such treatment.

Preferably said compound that is administered to treat autism is tazarotene.

### DETAILED DESCRIPTION OF THE INVENTION

The present invention provides a method for reducing the symptoms of autism in a patient, e.g. a human patient. Briefly, the method comprises administering a therapeutically-effective amount of tazarotenic acid or an acid or ester thereof, e.g. a lower alkyl ester of tazarotenic acid, to a human patient in sufficient quantities to reduce the effects of the autistic disease.

Preferably, the compound utilized in the method of the present invention is selected from the group consisting of tazarotene, i.e. ethyl-6-[2-(4,4-dimethyl-thiochroman-6-yl)ethyl]nicotinate, tazarotenic acid and other lower alkyl esters of tazarotenic acid, e.g. C₁-C₆ alkyl esters of tazarotenic acid, such as methyl 6-[2-(4,4-dimethyl-thiochroman-6-yl)ethyl]nicotinate, i-propyl 6-[2-(4,4-dimethyl-thiochroman-6-yl)ethyl]nicotinate, n-butyl 6-[2-(4,4-dimethyl-thiochroman-6-yl)ethyl]nicotinate, etc.

The administration of tazarotenic acid, salt or ester thereof provides a significant number of the patients with a significant reduction of one or more symptoms of autism, such as increased eye contact, better enunciation and use of pronouns, less fatigue, singing a song for the first time with the melody and words together and the entire song understandable, playing with age appropriate friends for the first time, fewer tantrums, better sleep patterns, improved politeness and coordination, being more loving, acknowledging another individual's emotion, increased voice and word association, etc.

A “therapeutically-effective amount” of tazarotenic acid, salt or ester thereof indicates an adequate amount of the active substances to have a significant, externally observable effect on the patient. Thus, such a therapeutically-effective amount affects one or more of the characteristics in the patient without the need for special equipment to determine the effect. For example, a therapeutically-effective amount of tazarotenic acid, salt or ester thereof has a significant, externally observable reduction of one or more of the symptoms of autism in a human patient without the need for special equipment to determine the effect. Accordingly, one can determine whether an adequate amount of the tazarotenic acid, salt or ester thereof has been administered by watching the patient and observing whether changes have occurred in the patient.

The therapeutic amount will vary with the potency of each of tazarotene acid, or ester, or salt, thereof, the amount required for the desired therapeutic effect, the rate of elimination or breakdown of the substance by the body once it has entered the bloodstream, and the amount of tazarotene acid, or ester, or salt, thereof in the formulation. In accordance with conventional prudent formulating practices, a dosage near the lower end of the useful range of a particular agent is usually employed initially, and the dosage is increased or decreased as indicated from the observed response, as in the routine procedure of the physician.

Preferably, the tazarotene acid, ester or salt, thereof, is administered in a manner to provide a maximum blood concentration of the active compound in a human or other animal of greater than 30 ng/ml, more preferably greater than 100 ng/ml. This concentration may be effected by ingestion by the patient of one or more of the capsules described in Table 1 of U.S. Pat. No. 6,656,500 B2, which is hereby incorporated by reference in its entirety.

Turning to a more detailed discussion of the invention, in a first aspect the present invention provides a method to reduce the symptoms of autism in a human patient. For example, the method of the invention reduces one or more symptoms, such as increased eye contact, better enunciation and use of pronouns, less fatigue, fewer tantrums, better sleep patterns, improved politeness and coordination, and increased voice and word association. In other words, the tazarotenic acid, salt or ester thereof is able to effect an adequate reduction of one or more of the observable characteristics of autism by an amount that is observable to a human observer, such as a parent, physician or caretaker, without the use of special devices such as microscopes or chemical analytical devices. The tazarotenic acid, salt or ester thereof reduces such symptoms by providing a therapeutically-effective amount of the active substance, and is administered in a composition which comprises also at least one of the group consisting of a physiologically acceptable carrier, adjuvant, excipient, buffer and diluent, which terms are used in their ordinary sense to indicate substances that assist in the packaging, delivery, absorption, or, in the case of an adjuvant, enhancing the therapeutic effect of tazarotenic acid, salt or ester thereof.

The physiologically acceptable carriers, adjuvants, excipients, buffers and diluents are nontoxic to recipients at the dosages and concentrations employed. Representative samples include water, isotonic saline solutions that are preferably buffered at physiological pH (such as phosphate-buffered saline or Tris-buffered saline), mannitol, dextrose, glycerol, and ethanol, etc. The carrier, adjuvant, excipient, buffer, or diluent may be combined with the tazarotene acid, ester or salt thereof to provide compositions either as liquid solutions or, in solid form. For example, when the compositions are to be administered orally, the compositions may be produced in any of powder, tablet or capsule form.

It may be advantageous to administer the tazarotenic acid, or ester, or salt, thereof utilizing a method of a slow release, for instance by intravitreal injection of the dose of tazarotenic acid, or ester, or salt, thereof encapsulated in a microvesicle, such as a liposome, from which the dose is released over the course of several days, preferably between about 3 to 20 days. Alternatively, the drug can be formulated for slow release, such as by incorporation into a slow release polymer from which the dosage of drug is slowly released over the course of several days, for example from 2 to 30 days. The slow release formulation may be placed in the eye by site-selective injection, i.e. by intravitreal, subconjunctival, periocular, intrascleral or subretinal injection. The tazarotenic acid, or ester, or salt, thereof may be incorporated into a bioerodible polymer such as a polylactic acid-glycolic acid copolymer, e.g. Oculex®.

The compositions administered according to the method of the present invention may be administered orally, but may also be administered via other direct routes, such as rectal or, in the case of pharmaceutically designed compositions, via transcutaneous methods such as intraarterial, intramuscular, intraperitoneal, subcutaneous, intraocular, and intravenous. Other routes such as buccal/sublingual, nasal, topical (such as transdermal and hypothalamic), vaginal and pulmonary may also be used, if desired. In the method of the present invention, the compositions are typically administered to human beings, but may also be administered to animals, preferably mammals, displaying symptoms similar to autism.

The invention is further illustrated by the following examples which are illustrative of specific modes of practicing the invention and are not intended as limiting the scope of the appended claims.

### EXAMPLE

A patient, diagnosed as autistic, and suffering from one or more of the symptoms of autism selected from the group consisting of eye contact avoidance, failure to socialize, attention deficit, poor mood, hyperactivity, anxiety, poor comprehension, inappropriate speech, abnormal sound sensitivity, poor digestion, disrupted sleep and perseveration is administered tazarotene in one or more capsules prepared according to Table I of U.S. Pat. No. 6,656,500 B2 to obtain a blood concentration of about 100 ng/ml. It is noted that a substantial number of said symptoms are improved upon obtaining said blood concentration.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

We claim:

1. A method of treating autism comprising administering a therapeutically effective amount of a compound selected from the group consisting of tazarotenic acid, or ester or salt, thereof, to a person in need of such treatment.

2. The method according to claim 1 wherein said compound is tazarotene.

3. The method according to claim 1 wherein said compound is administered orally.

4. The method according to claim 1 comprising rectally administering said compound in the form of a suppository.

5. The method according to claim 1 comprising parenterally administering said compound.

6. The method according to claim 5 wherein said parenteral administration comprises transdermal application of said compound and a pharmaceutically acceptable carrier.

7. The method according to claim 6 wherein said parenteral administration comprises sublingual application of said compound.

8. The method according to claim 1 wherein said compound is administered to a child in need of such treatment.

9. The method according to claim 1 comprising administering said compound at least once a day to obtain a maximum blood concentration of greater than 30 ng per ml.

10. The method according to claim 1 comprising administering said compound by site-selective injection into the eye of a slow release formulation comprising said compound incorporated in a bioerodible polymer.

11. A method of reducing the symptoms of autism in a human patient, comprising orally administering to the patient a composition comprising a therapeutically effective amount of a compound selected from the group consisting of tazarotenic acid, or ester or salt, thereof, and at least one of the group consisting of a physiologically acceptable carrier, adjuvant, excipient, buffer and diluent, wherein the composition is able to decrease the incidence of one or more symptoms of autism selected from the group of symptoms consisting of eye contact avoidance, failure to socialize, attention deficit, poor mood, hyperactivity, anxiety, poor comprehension, inappropriate speech, abnormal sound sensitivity, poor digestion, disrupted sleep, and perseveration, and wherein the decreased incidence is measured relative to the incidence in the untreated individual.

12. The method according to claim 11 wherein said compound is tazarotene.

13. The method according to claim 11 wherein said compound is administered orally.

14. The method according to claim 11 comprising rectally administering said compound in the form of a suppository.

15. The method according to claim 11 comprising parenterally administering said compound.

16. The method according to claim 15 wherein said parenteral administration comprises transdermal application of said compound.

17. The method according to claim 16 wherein said parenteral administration comprises sublingual application of said compound.

18. The method according to claim 11 wherein said compound is administered to a child in need of such treatment.

19. The method according to claim 11 comprising administering said compound at least once a day to obtain a maximum blood concentration of greater than 30 ng per ml.

20. The method according to claim 11 comprising administering said compound by site-selective injection into the eye of a slow release formulation comprising said compound incorporated in a bioerodible polymer.

21. A method of manufacturing a medicament able to reduce the symptoms of autism in a human patient, comprising combining a therapeutically-effective amount of a compound selected from the group consisting of tazarotene acid, or ester or salt, thereof, and at least one of the group consisting of a physiologically acceptable carrier, adjuvant, excipient, buffer and diluent, wherein the composition is able to decrease the incidence of one or more symptoms of autism selected from the group of symptoms consisting of eye contact avoidance, failure to socialize, attention deficit, poor mood, hyperactivity, anxiety, poor comprehension, inappropriate speech, abnormal sound sensitivity, poor digestion, disrupted sleep, and perseveration, and wherein the composition is suitable for oral administration and the decreased incidence is measured relative to the incidence in the untreated individual.
